(19) United States
(12) Patent Application Publication
(10) Pub. No.: US 2012/0271340 A1
(43) Pub. Date: Oct. 25, 2012

(54) Method and Apparatus for Embolic Protection During Heart Procedure

(75) Inventors: Thomas M. Castellano, Temecula, CA (US); Alexander J. Hill, Blaine, MN (US); H. Toby Markowitz, Roseville, MN (US)

(73) Assignee: Medtronic, Inc.

(21) Appl. No.: 13/093,212

(22) Filed: Apr. 25, 2011

(51) Int. Cl. A61F 2/01

(52) U.S. Cl. 606/200

(57) ABSTRACT

A method and apparatus is provided for embolic protection during a heart procedure to ablate an arrhythmia of the heart of the patient. The method may include placing an embolic protection filter and reshaping the mitral valve annulus.

### CROSS-REFERENCE TO RELATED APPLICATIONS

Cross-reference is hereby made to the commonly-assigned related U.S. application Ser. No. ______ (attorney docket number P0039491.01, entitled “Method and Apparatus for Treating a Mitral Valve Prolapse and Providing Embolic Protection”, filed concurrently herewith and incorporated herein by reference in it's entirety.

### FIELD OF THE INVENTION

This document relates generally to a medical device and more particularly to a method and apparatus to provide embolic protection during a heart procedure.

### BACKGROUND

Atrial fibrillation (AF) is a cardiac arrhythmia in which the atria, the upper chambers of the heart, quiver but do not pump blood by contracting forcefully or in an organized manner. AF is the most common sustained cardiac arrhythmia, affecting about 2.3 million in the United States and 4.5 million in the European Union. The disease which has an increasing prevalence with age is often associated with structural heart disease. Valvular disease and atrial dilatation are two conditions that may promote the initiation and/or maintenance of AF. Mitral valve prolapse, common in young women, is a condition in which the mitral valve may be thick, the chordae tendineae elongated, the mitral annulus dilated and the commissures not fused. AF is readily diagnosed from the electrocardiogram (ECG) with the absence of P waves and a regularly irregular ventricular rhythm. [ACC/AHA/ESC 2006 guidelines for the management of patients with atrial fibrillation, Circulation 2006; 114;e257-e354]

Patients with AF may experience an irregular and rapid heartbeat, heart palpitations, dizziness, sweating, chest pain, shortness of breath and, even, syncope. Patients with AF may be classified as paroxysmal, persistent or permanent. If paroxysmal, AF occurs suddenly and self-terminates or terminates by a maneuver executed by the patient. AF, if persistent, may be terminated by cardioversion, either chemical or electrical. In the third classification, permanent, AF can not be terminated by chemical or electrical cardioversion (discussed below). Brief paroxysms of AF while possibly symptomatic are not a cardiovascular concern. Patients with prolonged AF, however, are at risk of thromboembolic complications, the foremost of which is stroke. Patients with persistent or permanent AF must be protected from the risk of stroke.

Management of patients with AF consists of providing embolic protection and selecting one of two rhythm approaches. Maintenance of the atria in sinus rhythm, the normal rhythm of the heart, is termed rhythm control. Sinus rhythm refers to a rhythm originating near the sinus node, a region that is high in the right atrium. Allowing the atria to fibrillate is termed rate control. If sinus rhythm is restored, long-term embolic protection may not be needed. Although maintenance of sinus rhythm may be attempted via medication, such a strategy is often not effective. If sinus rhythm is abandoned and the atria are left to fibrillate, medication can be effective to limit (ventricular) heart rate. For those in whom control of heart rate with medication is not satisfactory or not tolerated, interruption of electrical communication from the atria to the ventricles may be accomplished with electrical ablation of the AV node and installation of a ventricular pacemaker to maintain a satisfactory heart rate.

Restoration of sinus rhythm in patients with AF, rhythm control, may be accomplished via an intervention termed cardioversion. Chemical cardioversion utilizes antiarrhythmic medication to convert the rhythm from AF to sinus rhythm. Electrical cardioversion of AF is the administration of an electrical shock typically across the patient's chest via electrode paddles or electrode patches, in a manner similar to defibrillation. Defibrillation utilizes paddles or patches attached to a patient's chest and administration of a large electrical shock. Cardioversion also applies an electrical shock, however, timed to follow electrical depolarization of the ventricles whereas defibrillation is delivered asynchronously. Effectiveness of electrical cardioversion is immediately obvious. The rhythm either returns to sinus or the atria continue to fibrillate. Chemical cardioversion, on the other hand, may require more than one hour before efficacy becomes apparent. Causes of AF may include electrophysiological abnormality of the atria, elevated atrial blood pressure, ischemia of atrial muscle, inflammatory or infiltrative disease of the atria, drug use and endocrine disorders. If the predisposing factors that contribute to the occurrence of AF are not removed, AF may return following cardioversion.

An alternative to restoring sinus rhythm via cardioversion is ablation. Catheter ablation aims to modify heart tissue to achieve a permanent cessation of AF. The application of energy delivered through catheters directly to the heart has seen widespread adoption and an evolution of techniques. Following the discovery of Hassaguerre that rapid firing in the pulmonary veins, vessels leading from the lungs to the left atrium, may lead to or be responsible for AF, catheter ablation has grown in use [Hassaguerre, et al. New England Journal of Medicine 1998:339-659-666]. Catheter ablation refers to techniques of tissue modification utilizing a catheter threaded into or on the heart. Cardiac tissue may be modified via a number of techniques, the most prominent being delivering of radio frequency energy but also including cryogenic cooling and delivering microwave energy. Catheters are inserted in or around the heart and a dose of the tissue modification therapy applied to change the heart with the desired outcome that AF will no longer occur.

The ablation procedure modifies atrial tissue to prevent the recurrence of AF and to maintain a patient in sinus rhythm, the normal rhythm originating in the upper chambers of the heart. The ablation procedure lasts about two hours but may range from 1 to 8 hours. Patients who are candidates for an ablation undergo various diagnostic procedures beforehand including the determination of the patient's risk for an embolus and imaging of the patient's pulmonary venous anatomy.

A mass formed from clotting is a thrombus. If the thrombus moves, it is said to have embolized and is called an embolus. AF frequently results in formation of a thrombus in the atrial appendages, areas of low and stagnant blood flow during AF. In such patients, restoration of the pumping function of the atria, such as occurs when the normal rhythm of the heart, sinus rhythm, is restored may result in the atrial appendages dislodging a thrombus. The result is embolization.

Pre-procedure imaging such as by computed tomography (CT) provides an understanding of patient specific pulmonary venous anatomy. CT is a diagnostic imaging modality that uses multiple sequential x-ray scans of the body to construct three-dimensional images. The pulmonary veins that transfer blood from the lungs to the left atrium of the heart are often involved in AF and are targets for ablation. A pre-procedure CT helps guide the physician during the ablation procedure in navigation and exploration of the left atria.

Transesophageal echo (TEE) may be used to determine whether thrombus exists in the left atrium. In this procedure, the patient is sedated or anesthetized. A small probe is inserted into the patient's mouth or nose and threaded down the esophagus until the probe is adjacent to the atria. Via use of ultrasound echocardiography such as TEE, the heart chambers may be visualized and thrombus, if present, detected. Thrombus in the left atrium is of critical concern because, if dislodged, an embolus from the left atrium may reach important systemic organs including the brain and peripheral musculature. If a thrombus is detected, the patient may be treated with agents to lyse the thrombus before the subject undergoes an ablation procedure. The patient with thrombus is at risk of embolus, especially if the patient in prolonged atrial fibrillation converts to sinus rhythm. Anti-coagulation, a pharmacologic measure for patients in AF, may be stopped or modified before the procedure, to aid in managing the puncture wound, described below, created for access to the venous circulation. A reduction or termination of anti-coagulation therapy elevates a risk of stroke as it eliminates a protection mechanism.

If the patient has persistent AF, the physician may elect to cardiovert the rhythm to restore sinus rhythm, using chemical cardioversion before the procedure or electrical cardioversion before or within the ablation procedure. If the patient has permanent AF, cardioversion may be accomplished, in essence, by performing an ablation procedure. At some point in the procedure, sinus rhythm may return as heart conduction is modified and the heart no longer sustains AF. Such restoration of sinus rhythm causes the atria to immediately pump blood and risks the dislodgement of thrombus. If the patient has paroxysmal AF, the patient will often present to the electrophysiology laboratory in sinus rhythm. However, the patient may present to the laboratory in AF or various stimulation challenges used during the procedure may cause the heart to enter AF. If the heart rhythm becomes AF, the physician may cardiovert the patient to restore sinus rhythm or may ablate tissue causing the rhythm to convert to sinus rhythm or the physician may ablate tissue and then employ electrical cardioversion to restore sinus rhythm. If the patient has not had AF for a prolonged period of time, the likelihood of a thrombus forming is low.

Ablation to treat AF may target areas of the atria that are found to fire rapidly, areas found to have certain electrical signatures, or certain anatomic regions including the pulmonary veins and areas of tissue that between anatomic features that are obstacles to conduction. A variety of ablation technologies may be utilized including cryogenic cooling, radio frequency (RF) heating, and microwave heating. Each is used in a controlled fashion to block conduction by causing permanent damage to specific regions of atrial tissue such that each region is no longer capable of propagating action potentials. In the example of heating, the effect on the tissue is similar to cooking while cryogenic cooling causes tissue to freeze and, therefore, undergo permanent modification.

By blocking conduction, a heart arrhythmia may no longer occur, may occur less frequently or may occur in a way that is amenable to medical management. Energy is applied such that the targeted portion of target organs are affected while surrounding tissues and organs are unaffected.

As the atria do not pump during AF, blood can stagnate in the atrial appendages leading to the formation of thrombus, a blood clot. Dislodgement of such thrombus creates an embolus from either the right or left atria. From the right, circulation leads from the heart to the lungs via the pulmonary artery with a pulmonary embolus as a possible result as the clot lodges in the lungs. Blockage of an artery in the lungs may cause significant symptoms requiring treatment typically with anticoagulation medication, although, severe cases may require surgical intervention. From the left heart, the circulation leads to the coronary and systemic circulations. An embolus from the left side of the heart may travel through the aorta to various organs including the brain where an embolus traveling through a carotid artery will likely cause a blockage in the brain resulting in a stroke. The clinical impact of stroke is devastating to patient, family and a burden on society, especially if recovery is incomplete.

Patients diagnosed with sustained AF are placed on anticoagulation medication to prevent the occurrence of stroke. By use of anticoagulation, a blood thinning agent, patients may live with AF for years without suffering neurologic consequences. However, patients not treated with anticoagulation are at significant risk of stroke.

Efforts to terminate AF are accompanied by an embolic risk, especially in patients who have had AF for a long duration as thrombus may have formed during the AF and then be dislodged when AF ceases. Resumption of sinus rhythm brings a sudden resumption of atrial contractions with the possibility to dislodge thrombus. Resumption may be spontaneous, by cardioversion or be the result of an ablation intervention which also presents thromboembolic risk [Schwarz, et al. Neuropsychological decline after catheter ablation of atrial fibrillation, Heart Rhythm 2010;7:1761-1767]. In patients who are to undergo an ablation procedure, diagnostic procedures are commonly undertaken to determine whether a thrombus is present in the atria. If a thrombus is detected, the patient is placed on medication to lyse the thrombus and the diagnostic procedure repeated until thrombus is not present at the time of ablation. Despite these precautions, a risk of thrombus dislodgement remains with cardioversion [Missault, et al. Embolic stroke after unanticoagulated cardioversion despite prior exclusion of atrial thrombi by transoesophageal echocardiography, European Heart Journal (1994) 15, 1279-1280]. While long-term anti-coagulation may not be needed, it is critically important in the setting of acute cardioversion.

In addition to pharmacological methods, a variety of apparatus to provide acute and chronic embolic protection have been proposed including the placement of blood filters downstream of the expected source of thrombus formation or dislodgement. Exemplary apparatus disclosed in U.S. Pat. No. 6,692,513 by Streeter, et al. include an apparatus for filtering and entrapping debris in the vascular system of a patient, wherein the filter captures debris carried in a blood flow. The filter mesh is sized so that it will pass blood therethrough but not debris, have a modest resistance to blood flow, and have a pore size of between about 40 microns and about 300 microns. U.S. Pat. No. 6,371,970 by Khosravi, et al. discloses an apparatus for filtering emobli from a vessel such as the ascending aorta, wherein a vascular device includes a support hoop with a blood permeable sac affixed to the support hoop and the hoop having regions that prevent material escaping from the sac when collapsed for removal. U.S. Pat. Pub No. 2007/0073333 by Coyle discloses a filter configured to protect against atheroembolization in a blood vessel including a region of a wire predisposed to form a laterally expanded shape when extended. And, U.S. Pat. Pub. No. 2006/0282114 by Barone discloses temporary prevention of embolization in a human blood vessel comprising a body transformable between a radially collapsed configuration and an expanded configuration sized and shaped for sealing against an inner wall of the vessel to obstruct fluid flowing therethrough. The application of Barone discloses a porous membrane that allows blood but not particulate debris to flow through the pores. All of the foregoing incorporated by reference in their entirety.

Mitral valve prolapse, a disease of the bi-leaflet valve between the left atrium and the left ventricle, has been associated with a high incidence of AF. Patients with mitral valve prolapse may present with distortion of the mitral valve annulus leading to incompetence of the mitral valve. During left ventricular contraction, the leaflets prolapse into the left atrium resulting in mitral regurgitation. With reguritant blood flowing retrogradely through the mitral valve, blood pressures in the left atrium are abnormally high, the left atrium distends and the occurrence of AF is more common. A variety of structural remedies have been proposed to improve the diseased mitral valve. Exemplary apparatus is disclosed in U.S. Pat. No. 6,793,673 by Kowalsky, et al. describing a mitral valve therapy device, positioned within the coronary sinus adjacent the mitral valve annulus and deployed. U.S. Pat. No. 6,702,826 by Liddicoat, et al. discloses constricting tissues to reduce the overall circumference of a valve annulus. U.S. Pat. Pub. No. 2008/0140188 by Randert, et al. discloses devices sized and configured to be positioned in a left atrium above the plane of a native mitral heart valve annulus to affect mitral heart valve function. U.S. Pat. No. 6,726,717 by Alfieri, et al. discloses an annular prosthesis for a mitral valve. And, U.S. Pat. Pub No. 2004/0019377 by Taylor et al. discloses a device for reducing mitral regurgitation with an elongated body positioned in a coronary sinus of a patient in a vicinity of a heart mitral valve to improve leaflet coaptation.

The management and the treatment of AF include a focus on reducing thromboembolic risk and a return, where practical, to sinus rhythm. Return to sinus rhythm, especially in a setting of a catheter ablation procedure places patients at elevated thromboembolic risk likely since much of the invasive catheter manipulation is done within the left atrium. Mitral valve prolapse is a co-existing condition for many patients with AF and the mitral valve is proximal to the left atrium where the electrophysiological interventions for eliminating AF are conducted. Apparatus and methods are needed to treat AF, reduce or eliminate mitral valve prolapse and provide distal embolic protection. Solutions are disclosed.

### SUMMARY

Ablating heart tissue of a patient may be used for the beneficial medical effects of modifying the properties of a tissue to treat a heart arrhythmia. Placing and retrieving an embolic protection filter in the patient downstream of the procedure site may reduce the risk of stroke due to dislodgement of thrombus or other matter. The filter may be placed prior to a cardioversion of the patient during the procedure. Exemplary embodiments provide for a delivery system, a filter to be delivered via the delivery system, and an apparatus to reshape the mitral valve.

### BRIEF DESCRIPTION OF THE DRAWINGS

FIG. 1 is a schematic illustration of a patient with a catheter in a femoral vein, the catheter extended into the heart of the patient;

FIG. 2 is a schematic illustration of a right atrium and a left atrium of the patient;

FIG. 3 is a schematic illustration of a right atrium and a left atrium of the patient, and a catheter having been extended through the right atrium, the interatrial septum and into the left atrium of the patient;

FIG. 4 is a top view of a delivery system incorporating a steerable catheter, a handle, a control knob and an inner catheter;

FIG. 5 is a perspective view of an embolic protection filter;

FIG. 6 is a schematic illustration of a mitral valve;

FIG. 7 is a schematic illustration of a right atrium and a left atrium of the patient, with the embolic protection filter;

FIG. 8 is a side view of the embolic protection filter;

FIG. 9 is a side view of the embolic protection filter as viewed from within the delivery system;

FIG. 10 is a schematic illustration of a right atrium and a left atrium of the patient, an embolic protection filter and a catheter having been advanced over a tether connected to the filter;

FIG. 11 is a schematic illustration of the expanded embolic protection filter;

FIG. 12 is a schematic illustration of the embolic protection filter having been partially collapsed;

FIG. 13 is a side view of an alternative embodiment of the embolic protection filter;

FIG. 14 is a side view of an alternative embodiment of the embolic protection filter;

FIG. 15 is a schematic illustration of a right atrium and a left atrium of the patient, an embolic protection filter is connected to a cooling source;

FIG. 16 is a schematic illustration of a mitral valve and a coronary sinus vein;

FIG. 17 is a schematic illustration of a stiffening member;

FIG. 18 is a schematic illustration of a right atrium and a left atrium of the patient and the shaping member inserted in the coronary sinus vein; and

FIG. 19 is schematic illustration of a right atrium and a left atrium of the patient, the shaping member inserted in the coronary sinus vein and an embolic protection filter placed in the left atrium.

### DETAILED DESCRIPTION OF THE INVENTION

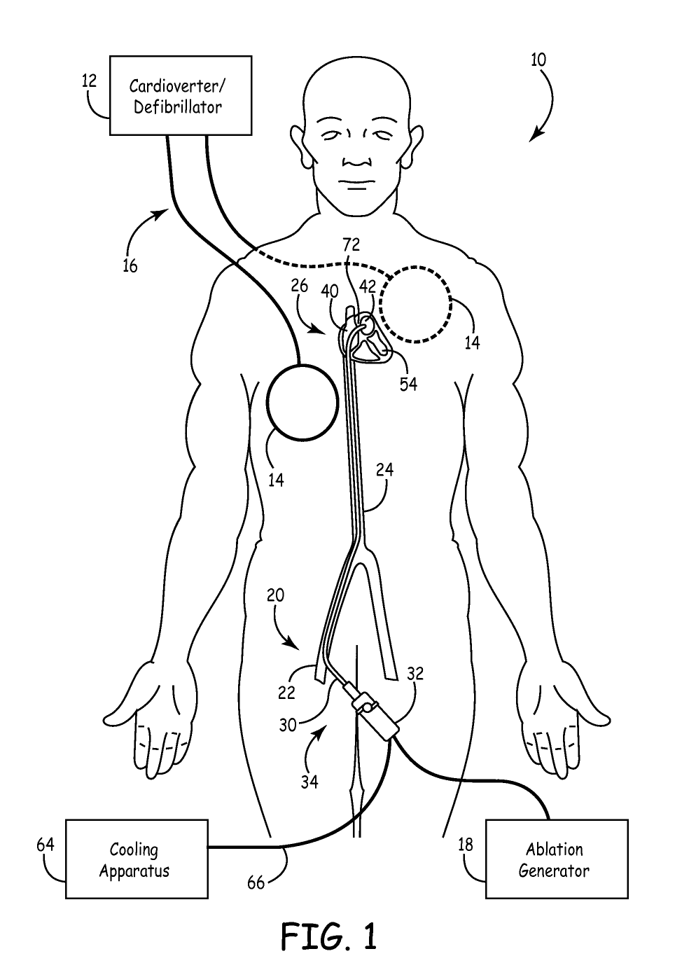

For an ablation procedure, electrode patches 14 are applied to patient 10 (see FIG. 1) for cardioverter/defibrillator 12 and electrodes (not shown) are also applied for electrocardiographic monitoring. Cardioverter/defibrillator 12 is attached via cables 16 to patches 14. Other physiological instrumentation is established such as plethysmography (not shown) for monitoring blood oxygenation and pulse rate of the patient. Cardioverter/defibrillator 12 can be used if the patient develops a life-threatening arrhythmia during the procedure and can also be used to convert patient 10 from AF to sinus rhythm. Patient 10 may be anesthetized with conscious sedation or general anesthesia and monitored appropriately.

A physician conducting an ablation procedure monitors a physiological condition of patient 10 including the patient's vital signs, the depth of anesthesia and patient electrophysiology by monitoring blood pressure, blood oxygen saturation level and electrograms on various instruments proximate to the physician.

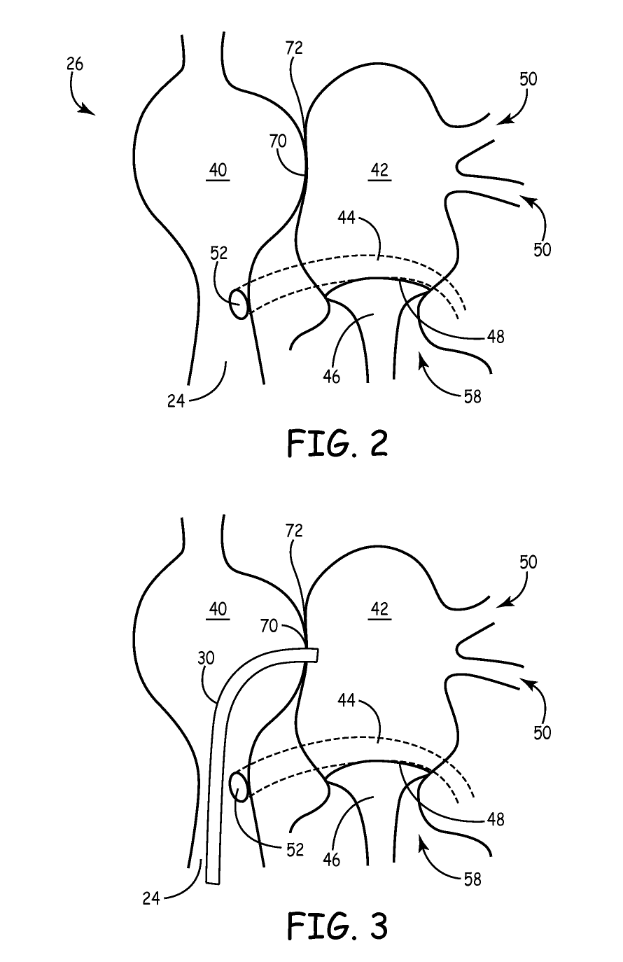

Access to the circulation of patient 10 is commonly through groin 20 although other areas of the body may be utilized such as the neck or arm. Right femoral vein 22, commonly used for access during electrophysiology procedures, is relatively large and easy to locate. Right femoral vein 22 is punctured percutaneously with a needle (not shown). An introducer with hemostasis valve (not shown) such as described in U.S. Pat. No. 5,843,031 issued to Hermann, et al. and incorporated herein in its entirety, is then inserted within the vein. A transeptal sheath is inserted and advanced through inferior vena cava 24 and into left atrium 42. Exemplary instruments that may be advanced are catheters as may be utilized for the electrophysiology procedure and for delivery of a filter, described below. Alternatively, access to the circulation of patient 10 may be through an artery, however, this is less commonly utilized. Access may also be obtained from the neck or arm (not shown). Access through vein 24 allows threading catheter 30 to inferior vena cava (IVC) 24 and to heart 26 (see also, FIG. 2).

With catheter 30 in right atrium 40 (FIG. 2), procedures can be performed on the right side of heart 26. As AF ablation commonly requires placing catheters in left atrium 42, access for catheter 30 to left atrium 42 is obtained. The foramen ovale is a flap-like structure that is open for circulation between the left and right sides of the heart in utero. Upon birth the foramen ovale becomes fossa ovalis 70 and closes due to the blood pressure differential between blood pressure in left atrium 42 and right atrium 40. Due to the change in relative pressure between the two atria that occurs at birth, the foramen ovale closes after birth. In the first year of life the flap-like structure of fossa ovalis 70 fuses.

Fossa ovalis 70 generally, is relatively thin, easily located and easily punctured. After puncturing, performing a procedure and removing instruments that were placed, fossa ovalis 70 closes. Fossa ovalis 70 is on the interatrial septum 72, a structure that is common to left atrium 42 and right atrium 40. FIG. 3 illustrates catheter 30 introduced into left atrium 42 through fossa ovalis 70. Circulation from left atrium 42 flows to the left ventricle (not shown) and then to the coronary circulation (not shown) and the systemic circulation (not shown). Systemic circulation leads to major organs including the brain and the skeletal musculature. Care must be taken to ensure air is not introduced into the left heart as circulation of air to the brain has disastrous consequences of stroke and permanent neurological injury.

The electrophysiology procedure of ablation involves navigating instruments to specific locations within heart 26, making measurements to understand the electrophysiology of each location, and, if appropriate, modifying the electrophysiology of specific locations with advantageous effect to treat an offending arrhythmia. Navigation is often done with the aid of fluoroscopy, a real-time imaging modality using x-ray radiation and detection. The physician is typically presented with the display (not shown) of a variety of signals including the patient's electrocardiogram, electrograms, signals from electrodes within the body, and an arterial blood pressure.

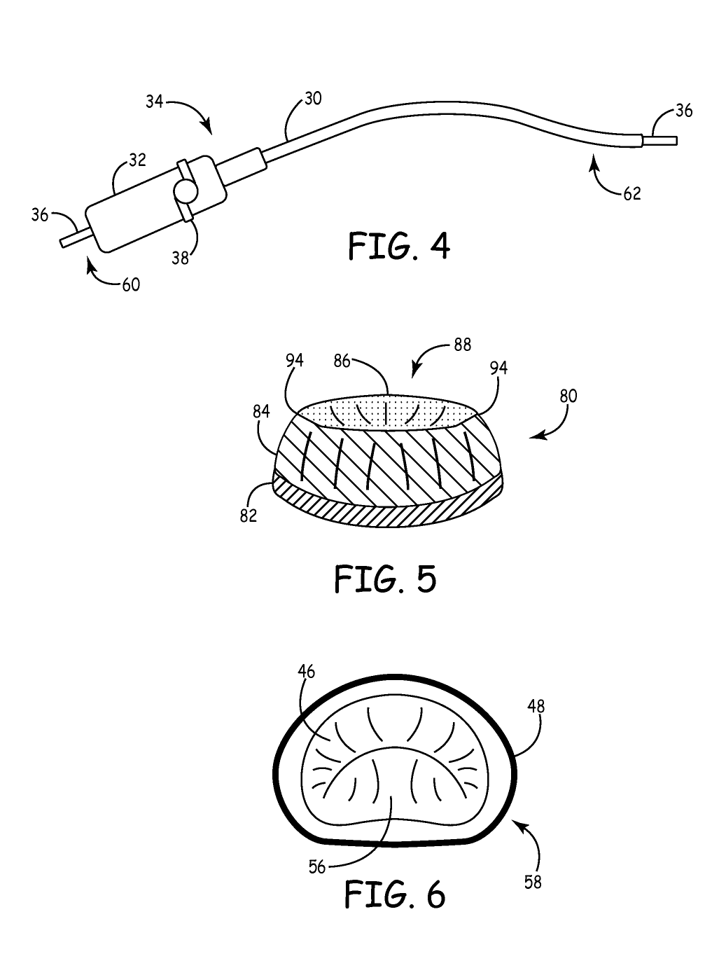

If the patient presents to the electrophysiology laboratory in AF, the physician may or may desire to convert the patient to sinus rhythm. If the patient's rhythm is to be converted to sinus, embolic filter 80 (see FIG. 5) is placed in patient 10 to protect patient 10 before the rhythm conversion. The conversion is called cardioversion and may be accomplished by a variety of techniques. In the electrophysiology laboratory, an expedient method is the use of DC cardioversion, the application of a large electrical shock synchronized to the beating of the heart's ventricles. The shock is administered under physician control from external cardioverter/defibrillator 12 connected to electrodes 14 on patient 10.

The electrocardiogram and electrograms from the patient are monitored during the electrophysiology procedure. During the procedure, various pharmacological agents may be delivered to the patient to aid in identification of areas for ablation and/or to test whether interventions have been effective. In addition, electrical stimuli may be applied to some of the various electrodes indwelling within the patient. The stimuli may be used to initiate the clinical arrhythmia, to test for conduction through various tissues of the heart or to terminate an arrhythmia that begins during the procedure.

Delivery system 34 shown in FIG. 1 and also in FIG. 4 incorporates steerable catheter 30, handle 32, control knob 38 and inner catheter 36. Delivery system 34 has proximal end 60 and distal end 62. Steering of catheter 30 is accomplished via control knob 38 that rotates about an axis defined by the center of circular knob 38 cross-section and is slideably connected to handle 32. Steering of catheter 30 is via pull wires, push wires, and the like within delivery system 34. Inner catheter 36 is introduced to proximal end 60 of delivery system 34 leading to a lumen of steerable catheter 30. Feeding inner catheter 36 through delivery system 34 results in inner catheter 36 exiting steerable catheter 30 at distal end 62. Delivery system 34 is operable to carry various tools (not shown) to puncture interatrial septum 72, electrode catheters for exploration and ablation, to measure intravascular pressure, to perform a biopsy, to deliver and to implant devices in heart 26 and to deliver and retrieve filter 80 (see FIG. 5). Elongated tubes 30, 36 of delivery system 34 are made of rubber, silicone or a polymer.

Embolic protection filter 80 traps particulate matter, especially thrombus that may have been produced during a prolonged period of AF. Filter 80 also traps matter that may result from the intervention, from the application of energy for ablation, or bubbles that may be introduced or produced during the intervention. Filter 80 may capture a bubble, a particle, or an embolus. To allow delivery and recovery of filter 80 through catheter 30, filter 80 is collapsed and packaged within delivery system 34 for shipment to the medical use facility, for example, an electrophysiology laboratory located in a medical facility. Filter 80 is advanced through delivery system 34 by use of inner catheter 36 that is used to push filter 80 and to eject filter 80 from delivery system 34.

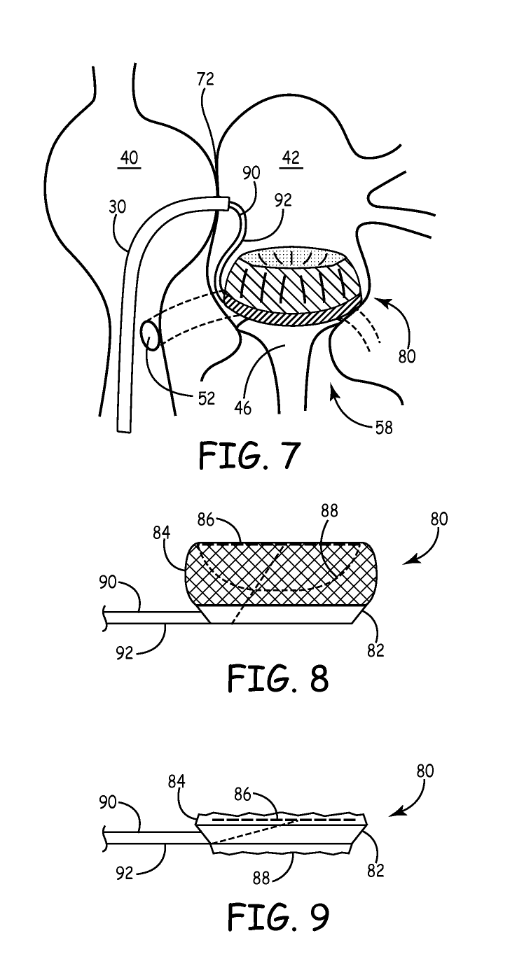

Filter 80 has annular ring 82 similar in shape to mitral valve annulus 48 (see FIG. 6). When deployed and delivered, filter 80 lies just above (upstream of) mitral valve annulus 48 (see FIG. 7). Filter 80 is positioned such that blood exiting the left atrium flows through filter 80 to capture a matter from the blood. Dome shaped porous mesh 84 is attached to annular ring 82. Dome shaped porous mesh 84 may be made from a porous membrane of sufficient strength to withstand compaction for delivery, subsequent expansion and re-compaction for retrieval as well as manipulation within heart 26. Mesh 84 may include therapeutic agents to facilitate particle capture and encourage thrombotic capitulation of blood that is in a pre-thrombotic condition. Annular ring 82 varies in shape as heart 26 changes shape during pumping. During diastole, annular ring 82 generally lies in a plane that is about parallel to the plane of mitral valve annulus 48. Annular ring 82 is made of materials that can withstand compaction for delivery, subsequent expansion and recompaction for retrieval as well as manipulation and flexing within heart 26. Materials used in construction of annular ring 82 may include nitinol and various polymers. See, for example, U.S. Pat. No. 6,692,513 column 6, lines 8-12 and U.S. Pat. No. 6,371,970 column 4, line 65 to column 5, line 8. Dome mesh 84, when deployed rises above ring 82, away from mitral valve annulus 48. Approximately midway from ring 82 to the center of dome mesh 84 lies stiffening member 86. Member 86 may use, for example, nitinol or other material having a shape memory. Member 86 follows the general outline as annular ring 82 with the exception of two diametrically opposed vertices 94 that facilitate collapsing filter 80 and preparing it for insertion into the delivery system. The two vertices are aligned to facilitate collapsing for entry into delivery system 34. Annular ring 82 and stiffening member 86 are radiographically opaque and visible on fluoroscopy permitting assessment of filter 80 orientation.

Concave portion 88 of dome mesh 84 is defined at its periphery by stiffening member 86. When delivered, all of concave portion 88 is between stiffening member 86 and annular ring 82. Concave portion 88 of filter 80 is above and does not touch mitral valve leaflets, anterior leaflet 56 (FIG. 6) and posterior leaflet 46 (FIGS. 2, 6). Stiffening member 86 is attached to dome mesh 84 by stitching.

FIG. 8 illustrates two tethers connected to filter 80. Ring tether 90 is attached to annular ring 82 for retrieving filter 80 and drawing filter 80 into distal end 62 of delivery system 34. Tethers 90, 92 extend the length of delivery system 34 by at least two feet, in addition, to be available to for physician control and retrieval of filter 80. Dome tether 92 is attached to stiffening member 86 to collapse stiffening member 86 and to retain trapped filter material during retrieval of filter 80. Dome tether 92 and ring tether 90 and junctions of the tethers are advantageously coated with anti-thrombotic materials such as or similar to streptokinase, tissue plasminogen activator or the like to discourage formation of a thrombus during an intervention. Tethers 90, 92 are exemplary woven and constructed of strands, fibers, filaments, or the like using materials such as Teflon, or other polymers, exemplary used in the construction of a ligature.

FIG. 9 illustrates filter 80 as reformed and compressed as it would appear inside inner catheter 36. Concave portion 88 of dome mesh 84 is folded such that stiffening member 86 is above (upstream) annular ring 82 and concave portion 88 of dome mesh 84 is below (downstream). Upon expulsion from inner catheter 36 in delivery system 34, filter 80 achieves the shape illustrated in FIG. 8, annular ring 82 and stiffening member 86 expand applying tension to dome mesh 84. Filter 80 expands in heart 26.

Filter 80 traps bubbles such as might be formed by the introduction of a gas into heart 26 with the introduction of instruments into the heart or during the application of energy to perform the ablation. Filter 80 also traps particulate matter such as thrombus that may form upstream of filter 80 or matter produced in performing the ablation. Filter 80 retains the trapped matter while filter 80 is being positioned, after having been positioned and while filter 80 is being retrieved, described below.

In another embodiment, trapping matter such as thrombus allows the matter to be lysed by biological mechanisms inherent in the bloodstream. Maintaining the trapped matter upstream of the mitral valve ensures the matter does not flow to key body organs and allows time for the lysing activity of blood flow to act upon and dissipate the matter. Filter 80 can also be cooled via cooling apparatus 64 (see FIG. 1) to expedite the lysing of the matter, especially in the setting of the delivery of heat to the tissue and the blood while performing an ablation. Delivery system 34 is fluidly coupled to filter 80. Annular ring 82 is hollow to allow transport of a fluid or a gas and also allowing ring 82 to be inflated with a gas such as nitrogen and carbon dioxide or a liquid such as saline or to be cooled by cooling apparatus 64. By controlling the inflation of annular ring 82, the size and shape of filter 80 is expanded and adjusted in the heart

In another embodiment, filter 80 is constructed of resorbable materials, for example, as disclosed in U.S. Pat. Pub. 2010/0286758 [0025] and [0027], incorporated herein in its entirety, by reference. In this embodiment, annular ring 82, stiffening member 86 and dome mesh 84 are composed of resorbable materials. All other tethers and delivery members used to place filter 80 are withdrawn, either immediately following delivery of filter 80 or at the end of the ablation procedure. Resorbable filter 80 structure remains intact in heart 26 of patient 10 following the ablation procedure. Following the procedure, the structure is resorbed in patient 10.

In an alternate embodiment, portions of filter 80 are constructed of resorbable materials including stiffening member 86 and dome mesh 84. Annular ring 82, however, is made of a material that is biocompatible, is not resorbable and is intended for permanent implantation in patient 10. In the event that the arrhythmia for which patient 10 was being treated, returns following the ablation procedure, a second, subsequent ablation procedure may be performed. When the second ablation procedure is performed, annular ring 82 remains while the remainder of filter 80 has been resorbed. Annular ring 82 serves as a landing zone for deployment of a filter used in the second ablation procedure. Return of an arrhythmia is not uncommon, about 30% of patients who undergo AF ablation return for a subsequent ablation procedure.

Filter 80 captures particulate matter and bubbles that flow through and out of left atrium 42. In an alternative embodiment, delivery system 34 is directed from inferior vena cava 24 to right atrium 40, through the tricuspid valve (not shown), into the right ventricle and filter 80 is delivered to the right ventricular outflow tract (not shown), adjacent to the pulmonic valve (not shown). In this position, filter 80 traps matter and bubbles that flow through and out of the right ventricle (not shown) of heart 26.

Following the ablation of tissue, sufficient time is allowed for such matter to dislodge and be trapped by filter 80 before filter 80 is removed from patient 10. The time interval that is allowed for dislodgement and trapping following the ablation is at the discretion of the physician, typically 10 minutes or less and nominally 5 minutes. During this time and afterwards, filter 80 retains matter trapped in the filter and retains the matter during retrieval of filter 80. To remove filter 80, the filter is invaginated and closed to capture such matter and bubbles. Then, after the matter and bubbles are secured within the invaginated and closed filter 80, filter 80 is further collapsed and drawn into catheter 36 and removed from patient 10. In this manner, trapped matter and bubbles are retained and are not released into the bloodstream during retrieval and recovery of filter 80.

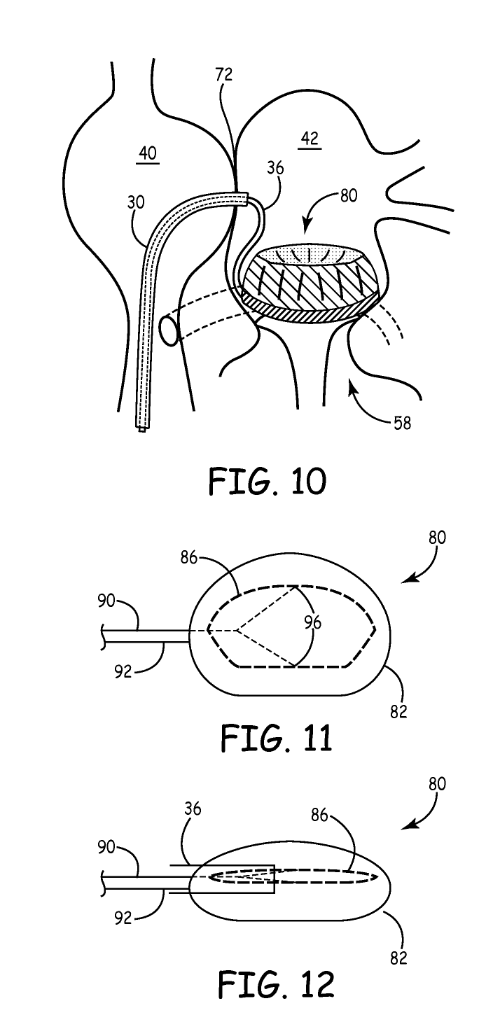

FIG. 7 shows filter 80 in position over mitral valve 58. FIG. 10 shows inner catheter 36 having been advanced over tethers 90, 92 to filter 80. The schematic illustration of FIG. 11 shows elements of filter 80 as viewed from above, dome tether 90, ring tether 90, stiffening member 86 and annular ring 82. From left to right, dome tether 90 bifurcates and attaches to stiffening member 86 in two locations 96 on the downstream side of dome mesh 84. In FIG. 12, inner catheter 36 has been advanced over tethers 90, 92, over the bifurcation of dome tether 90, causing stiffening member 86 to narrow, close and capture trapped debris and annular ring 82 to narrow. In addition, advancing inner catheter 36 over dome tether 90, forces stiffening member 86 to descend to just above annular ring 82 approximating the side view of filter 80 as shown in FIG. 9. Tension is then applied to ring tether 92, filter 80 is retracted to steerable catheter 30 and drawn into filter 80 by causing annular ring 82 to conform to the inner dimensions of catheter 30.

Filter 80 is shaped to occupy the space above the mitral valve and to cause all blood that is to exit the left atrium to flow through filter 80 by sealing filter 80 to the wall of left atrium 42 near or on mitral valve annulus 48. Filter 80 is shaped so particulate matter and bubbles flowing through left atrium 42 are directed to the concave portion 88 of filter 80 for later recovery.

Filter 80 is shaped so that in the setting of mitral valve prolapse, filter 80 does not interfere with prolapsing mitral valve leaflets 46, 56. The shape of filter 80 may be symmetric or asymmetric about an axis perpendicular to a plane containing annular ring 82.

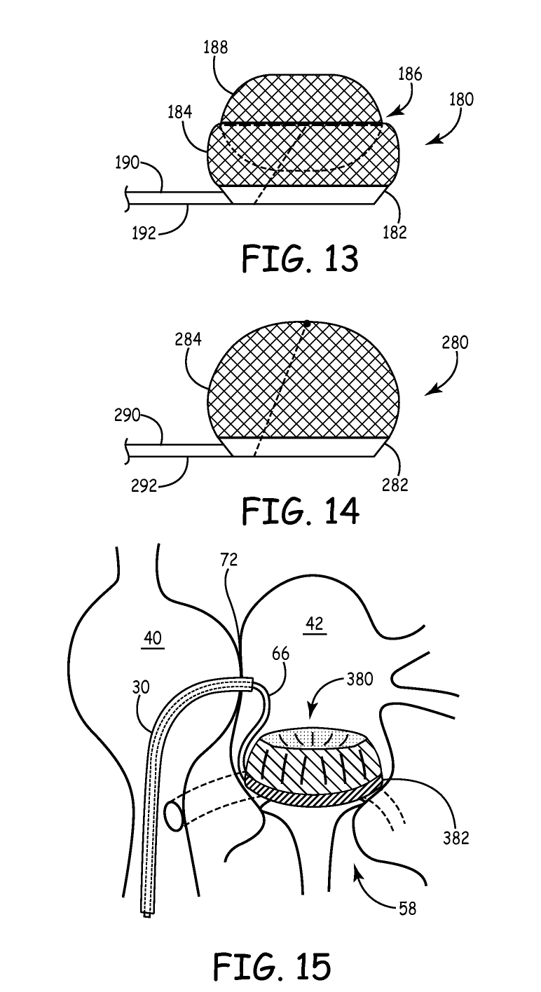

In an alternative embodiment shown in FIG. 13, filter 180 is deployed with a central portion of dome mesh 184, portion 188 being convex rather than concave as illustrated by portion 88 in FIG. 8. Dome mesh 184, stiffening member 186, ring tether 190, dome tether 192 and annular ring 182 correspond respectively to dome mesh 84, stiffening member 86, ring tether 90, dome tether 92 and annular ring 82 of FIG. 8. In the embodiment of FIG. 13, convex portion 188 of dome mesh 84 is folded such that stiffening member 86 is above (upstream) annular ring 182 and convex portion 188 of dome mesh 184 is above (upstream). Upon expulsion from inner catheter 36 in delivery system 34, filter 180 achieves the shape illustrated in FIG. 13.

Another embodiment shown in FIG. 14 illustrates filter 280 deployed without a stiffening member and taking a dome shape being partly spherical. Dome tether 192 is attached to dome mesh 284 as illustrated. Dome mesh 284, ring tether 290, dome tether 292 and annular ring 282 correspond, respectively, to dome mesh 84, ring tether 90, dome tether 92 and annular ring 82 of FIG. 13. Dome mesh 284 is sufficiently stiff so that upon expulsion from inner catheter 36 in delivery system 34, filter 280 achieves the shape illustrated in FIG. 14.

Filter 80 may be delivered and positioned as a first step after gaining access to the left atrium, it may be delivered in preparation before performing a cardioversion or it may be delivered near or at the conclusion of the ablation procedure. Filter 80 may be used acutely and retrieved, or it may be left in place. If left in place for later retrieval, tethers 90, 92 are severed, leaving remnant portions protruding a short distance into right atrium 40. Remnant portions of tethers 90, 92 may later be snared for recovery and retrieval of filter 80.

In an alternative embodiment, filter mesh 84 and stiffening member 86 are made of a resorbable polymer. Following the ablation procedure, filter 80 is left in heart 26 of patient 10. The resorbable materials are engineered to persist in the bloodstream for a period longer than the expected lysing of material trapped in filter 80.

After delivery system 34 is placed in left atrium 42, inner catheter 36 is advanced. Under fluoroscopy, filter 80 is viewed being ejected from inner catheter 36. As filter 80 leaves delivery system 34, annular ring 82 and stiffening member 86 are viewed expanding. Annular ring 82 is easily distinguished from the stiffening member as ring 82 is larger in diameter than member 86. Filter 80 is oriented so annular ring 82 is inferior to stiffening member 86. Although structural portions of heart 26 are only faintly visible on fluoroscopy, filter 80 is oriented by ensuring stiffening member 86 is closer to the head of the patient than annular ring 82. Filter 80 is unrestrained as it is given slack via tethers 90, 92 and allowed to float to mitral valve annulus 48. Blood flow from left atrium 42 through mitral valve 58 causes drag on filter 80, allowing filter 80 to center and position the filter 80 adjacent and just above mitral valve 58. In this manner, filter 80 is placed upstream of and proximal to mitral valve 58.

In another embodiment, annular ring 382 (FIG. 15) has small holes in its outward facing portion (not visible). Inner catheter 66 is fluidly coupled to annular ring 382 at its distal end and to a vacuum source, “suction” (not shown), at its proximal end, after filter 380 is positioned just above mitral valve annulus 48. The application of the vacuum ensures retention of annular ring 382 and filter 380 to the left atrial wall.

In an alternative embodiment, annular ring 382 (see FIG. 15) is coupled to cooling apparatus 64 (see FIG. 1) via fluid connection 66 and filter 380 is positioned just above mitral valve 58. Cooling apparatus 64 is activated to cool annular ring 382 to achieve cryoadhesion of annular ring 382 to the wall of left atrium 42 but not so cold as to create permanent tissue modification. Filter 380 and annular ring 382 are similar to and correspond to filter 80 and annular ring 82, respectively, of FIG. 8.

Filter 80 may be additionally secured to the heart wall via a clip or a staple (not shown) applied through delivery system 34. In a further embodiment, annular ring 82 is attached to mitral valve annulus 48 by use of sutures (not shown). The sutures, when pulled taught, ensure a sealing of annular ring 82 to the wall of left atrium 42 and ensure all blood from left atrium 42 flows through filter 80. In this embodiment, mitral valve annulus 48 may be made to conform to the shape of annular ring 82. Advantages of the conformation are described below.

During delivery and placement of filter 80 for the ablation procedure, the physician may choose to modify mitral valve annulus 48 shape. Conducting the modification concurrently with the ablation procedure does not require additional vascular access and may utilizes delivery system 34 that is already in place. The modification may be temporary, for the duration of the ablation procedure and then removed or, it may be a permanent modification, one that is left in place after the ablation procedure.

A variety of procedures and devices exist for restoring the normal geometry of mitral valve annulus 48 and the apposition of anterior leaflet 56 with posterior leaflet 46. In patients with chronic mitral regurgitation the abnormal geometry results in poor leaflet coaptation. Use of a remodeling ring and conforming mitral valve annulus 48 to the shape of the remodeling ring, annuloplasty, restores the normal size and shape of annulus 48. The annuloplasty ring helps prevent further annular dilatation while restoring leaflet movement to nearer normal by improving the coaptation. When reforming a mitral valve annulus to improve mitral valve performance, annular ring 82 is manufactured to the shape of a well-functioning valve, a desired shape for mitral valve annulus 48. Annulus 48 is made to conform to ring 82 by attachment where the attachment is one of a suture, a staple or a vacuum. Reforming a mitral valve annulus may also be implemented using a shaping member delivered to an adjoining structure, described below.

Patients undergoing an ablation procedure and who also have mitral valve prolapse may benefit from another embodiment in which annular ring 82 is attached to mitral valve annulus 48 in the manner described above, however, the sutures used to fasten the annular ring are tied and cut so as to effect a permanent implantation of filter 80. Dome mesh 84 and stiffening ring 86 are made of resorbable polymers. In this embodiment, dome mesh 84 and stiffening ring 86 gradually decompose over a period of days to weeks during which time the material trapped by the filter is lysed by the continuous flow of blood through filter 80.

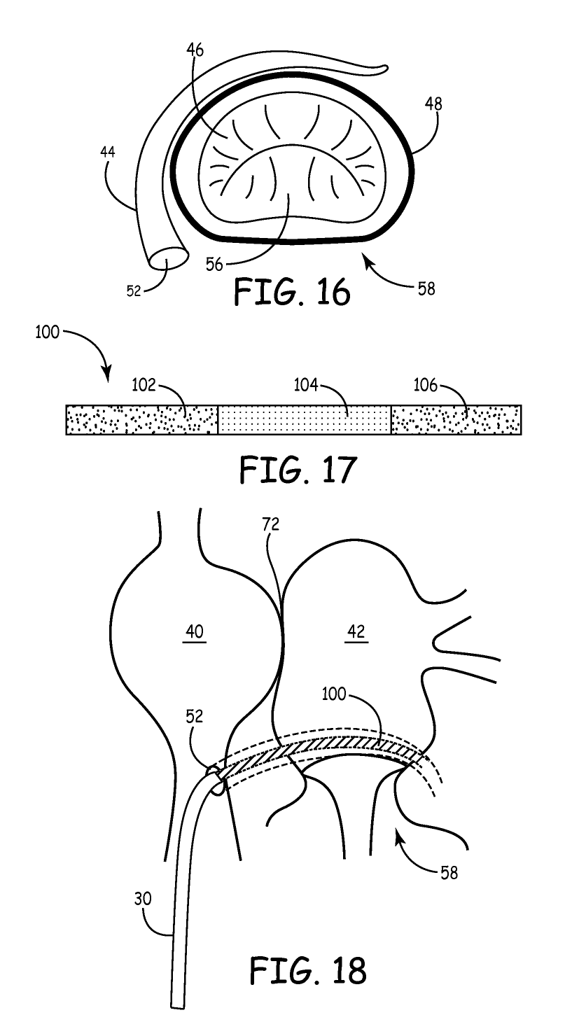

Delivery system 34 may be utilized to cannulate ostium 52 of coronary sinus 44 (see FIG. 16) and to deliver shaping member 100 (see FIG. 17) in coronary sinus vein 44 in the portion of vein 44 that is proximate to mitral valve annulus 48, thus delivering shaping member 100 to mitral valve 58. Placing shaping member 100 in coronary sinus vein 44 (FIG. 18) allows beneficial modification of mitral valve annulus 48 shape, particularly in patients with concomitant mitral valve prolapse. Shaping member 100 contains inner portion 104 that is stiff, ferromagnetic and has been magnetized. Placing shaping member 100, described above, improves apposition of mitral valve leaflets 46, 56. Shaping member 100 has portions 102, 106 that are relatively flexible compared to center portion 104. Shaping member 100 is delivered to vein 44 through catheter 30, the physician pushing inner catheter 36 to press against member 100 and to expel it from catheter 30 into vein 44. Shaping member 100 is secured to vein 44 via magnetic attraction to filter 480, described below, or may be secured via suction, a ligature, a staple or by wedging a distal end of distal portion 106 of member 100 in a narrow portion of coronary sinus vein 44 (see FIG. 16), distal to coronary sinus ostium 52.

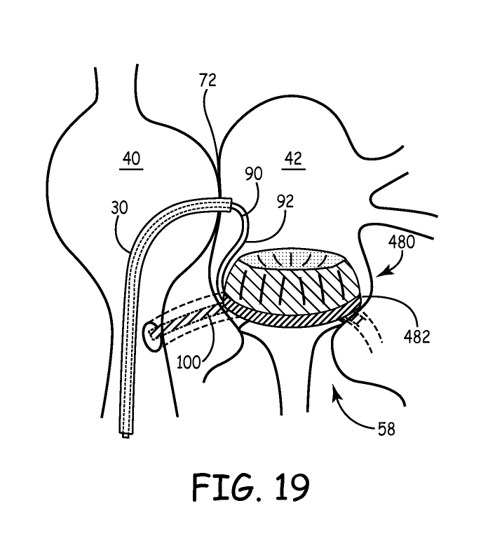

FIG. 19 shows an embodiment of embolic protection filter 480 constructed similarly to filter 80. Annular ring 482 is similar in shape and flexibility to annular ring 82 and like member 100, ring 482 has a ferromagnetic section. Two magnetized elements, annular ring 482 and member 100 are magnetized with polarities such that they are attracted when member 100 is in coronary sinus vein 44 and filter 480 is placed near mitral valve annulus 48. Delivery of filter 480 via delivery system 34 follows delivery and location of shaping member 100 to coronary sinus vein 44. Magnetic attraction between member 100 within coronary sinus vein 44 and annular ring 82 of filter 80 aids in alignment, retention and sealing of blood for embolic protection filter 480. Delivery of shaping member 100 reforms mitral valve annulus 48.

Ablation involves the use of various elements such as catheters to locate targeted tissue and to apply energy to cause the desired change in tissue conduction. To ablate a user administers ablation energy delivered from ablation generator 18 to heart 26 via electrodes on a catheter (not shown) delivered via delivery system 34. To cardiovert heart 26, the user administers cardioversion energy from cardioverter/defibrillator 12 to the body via patient electrodes 14. Cardioversion energy may also be delivered to heart 26 via electrodes (not shown) in or on heart 26 via delivery system 34 in patient 10.

Following cardioversion, as with ablation, a thrombus may not be dislodged immediately so it is appropriate to wait a period of time before recovering and retrieving filter 80. The period of time to wait may be as much as 10 minutes, however, nominally the waiting period is 5 minutes.

Following withdrawal of delivery system 34 and withdrawal of associated components from the vasculature of patient 10, attention is paid to closing the wound to prevent a loss of blood or a hematoma at the site of vascular access, right femoral vein 22.

We claim:

1. A method of providing embolic protection in a patient comprising:
inserting a steerable elongated member into a patient, the member having a proximal end and a distal end;
extending the distal end of the elongated member into a right atrium of a heart of the patient;
extending the distal end of the elongated member into a left atrium of the heart of the patient;
delivering an embolic protection filter to the heart via the elongated member;
placing the embolic protection filter in a blood exiting the left atrium of the heart; and
securing the filter in the heart of the patient.

2. The method of claim 1 wherein the filter is capable of being resorbed in the patient.

3. The method of claim 1 further comprising:
expanding or adjusting the filter in the heart of the patient.

4. The method of claim 3 further comprising:
coupling at least one of a gas and a liquid to the filter; and
expanding the filter by the liquid or the gas.

5. The method of claim 1 further comprising securing the filter to the heart with at least one of a blood flow, a suction, a ligature, a staple, a clip, and a cryoadhesion.

6. The method of claim 1 wherein the filter has an annular ring having about the same shape as a mitral valve annulus of the heart, and, being placed in a plane about parallel to the mitral valve annulus

7. The method of claim 1 wherein the filter is capable of capturing at least one of:
a bubble, a particle and an embolus.

8. The method of claim 7 wherein the filter is capable of retaining and retrieving at least one of: an embolus, a bubble, and a particle.

9. The method of claim 1 further comprising:
coupling a cooling apparatus to the filter via the elongated member; and
cooling the filter via the cooling apparatus and the elongated member.

10. The method of claim 1 further comprising:
treating a heart arrhythmia of the patient by at least one of:
coupling a cardioverter to the patient; and cardioverting the heart of the patient; and
coupling an ablation generator to the patient and ablating a heart tissue of the patient.

11. The method of claim 10 further comprising, in this order:
waiting a pre-determined period of time; and then,
retrieving the filter.

12. The method of claim 11 wherein the pre-determined time is about 10 minutes.

13. A delivery system for providing embolic protection in a patient with a heart arrhythmia comprising:
a steerable elongated member, the member having a proximal end and a distal end, the distal end being delivered through a right atrium to a left atrium of a heart of the patient; and
an embolic protection filter being delivered to a left atrium of the heart of the patient from within the elongated member, the filter being placed in a blood exiting the left atrium, being secured to the heart; and operable to capture at least one of: a bubble, an embolus and a particle.

14. The system of claim 13 wherein the filter is resorbed in the patient.

15. The system of claim 13 wherein the filter is operable to self-expand or to be expanded.

16. The system of claim 13, wherein the filter has an annular ring, the ring being placed in a plane about parallel to a mitral valve annulus and the annular ring being about the same shape as a mitral valve annulus.

17. The system of claim 13 wherein the filter is operable to capture at least one of:
a bubble, a particle and an embolus.

18. The system of claim 17 wherein the filter is operable to:
retain and retrieve at least one of: a bubble, a particle and an embolus.

19. The system of claim 13, further comprising:
a cooling apparatus, the cooling apparatus being coupled to the elongated member and to the filter, the filter being cooled by the cooling apparatus.

20. The system of claim 13 wherein the patient being coupled to at least one of a cardioverter and an ablation generator and the system operable to treat a heart arrhythmia of the patient via the cardioverter or the ablation generator.

21. The system of claim 13, wherein the filter is affixed to the heart by at least one of:
a blood flow, a suction, a ligature, a staple, a clip, and a cryoadhesion.

22. A system for embolic protection of a patient comprising:
means to filter a matter from a blood of the patient during a termination of a heart arrhythmia of the patient; and
means to retrieve the matter from the patient.
